(19) United States
(12) Patent Application Publication
(10) Pub. No.: US 2026/0026899 A1
(43) Pub. Date: Jan. 29, 2026

(54) CONTROL OF A SURGICAL ROBOT ARM

(71) Applicant: CMR Surgical Limited, Cambridge (GB)

(72) Inventor: Martin Blake, Cambridge (GB)

(73) Assignee: CMR Surgical Limited, Cambridge (GB)

(21) Appl. No.: 18/997,112

(22) PCT Filed: Aug. 15, 2023
(86) PCT No.: PCT/GB2023/052143

§ 371 Date: Jan. 20, 2025

(30) Foreign Application Priority Data
Aug. 15, 2022 (GB) 2211913.5

(51) Int. Cl. A61B 34/30; A61B 34/00; A61B 90/00

(52) U.S. Cl. CPC A61B 34/30; A61B 34/71; A61B 2034/305; A61B 2090/066

(57) ABSTRACT

A controller for moving a first part of a surgical robot arm, the surgical robot arm comprising a plurality of arm segments separated by a plurality of driven joints, in response to an external force being imparted on a second part of the surgical robot arm, the controller being configured to: determine a torque at each of the plurality of joints which results from the force imparted on the second part of the surgical robot arm; calculate from the determined torques a resultant force which acts on the first part of the surgical robot arm as a result of the external force being imparted on the second part of the surgical robot arm; calculate a desired velocity of the first part of the surgical robot arm for a time subsequent to a current time by evaluating an equation of motion modelling Coulomb and viscous frictional forces using a backward Euler approximation, the equation of motion having inputs of the calculated resultant force which acts on the first part of the surgical robot arm; and the current velocity at the current time of the first part of the surgical robot arm; and drive the surgical robot arm in accordance with the calculated desired velocity.

### BACKGROUND

It is known to use robots for assisting and performing surgery. FIG. 1 shows a typical surgical system 100 which comprises a base 101, a surgical robot arm 102 and an instrument 103. The base supports the surgical robot arm, and is itself attached rigidly to, for example, the operating theatre floor, the operating theatre ceiling or a trolley. The arm 102 extends between the base and the instrument. The arm is formed of multiple arm segments 105 and is articulated by means of multiple driven joints 104 between the arm segments which are used to position the surgical instrument 103 in a desired location and orientation relative to the patient. The driven joints 104 include the elbow 104e and the wrist 104w.

The surgical instrument 103 is attached to the distal end of the terminal arm segment 105t of the robot arm. During an operation, the surgical instrument can penetrate the body of a patient at a port so as to access the surgical site.

FIG. 1 illustrates that the surgical robot 100 forms part of a system also including a surgeon command interface 201 and a controller 202. The controller comprises a processor 203 and a memory 204. The memory 204 of the controller 202 stores in a non-transient way software that is executable by the processor 203 to cause the arm 102 to move as desired. The controller is coupled to actuators (not shown) in the surgical robotic arm. The actuators are coupled to the joints in the robot arm such that driving the actuators causes the angles at the joints to change, causing the arm to move. The controller 202 can therefore effect motion of the robot arm 102 in accordance with outputs generated by execution of the software. The controller 202 may be remote from the arm 102.

The software can control the processor 203 to drive the arm in dependence on inputs from a surgeon command interface 201, which may comprise one or more input devices whereby a user can request motion of the end effector in a desired way. The input devices could, for example, be manually operable mechanical input devices such as control handles or joysticks, or contactless input devices such as optical gesture sensors. The software stored in memory 204 is configured to respond to those inputs and the processor 203 is configured to execute the software to cause the joints of the arm to move accordingly.

When the surgical robot arm is in a compliant mode, the software can control the processor 203 to drive the arm in dependence on an external force acting on the arm 102 For example, the controller is able to cause the arm to move in response to a member of the bedside team pushing on a part of the arm, thereby giving the impression that the member of the bedside team is physically moving the arm. This functionality is useful in a number of scenarios including those in which a member of the bedside team is changing the instrument connected to the arm and those in which a member of the bedside team notices that a portion of the arm is going to collide with another piece of apparatus in the operating room. In this example, the member of the bedside team is able to push on a part of the robot arm so that the arm moves out of the way of the neighbouring apparatus.

The process of driving the arm in response to an external force being exerted on a part of the arm broadly involves gathering information about the external force which has been imparted on the arm and deciding, based on that force, how the arm should behave in response to that force. How the arm should behave in response to the force is dictated by a chosen impedance model, the properties of which may be chosen arbitrarily to achieve whatever behaviour of the arm is desired.

The present application concerns improved control of the movement of a surgical robot arm in response to an external force being imparted on the arm.

### SUMMARY OF INVENTION

According to a first embodiment, there is provided a controller for moving a first part of a surgical robot arm, the surgical robot arm comprising a plurality of arm segments separated by a plurality of driven joints, in response to an external force being imparted on a second part of the surgical robot arm, the controller being configured to: determine a torque at each of the plurality of joints which results from the force imparted on the second part of the surgical robot arm; calculate from the determined torques a resultant force which acts on the first part of the surgical robot arm as a result of the external force being imparted on the second part of the surgical robot arm; calculate a desired velocity of the first part of the surgical robot arm for a time subsequent to a current time by evaluating (i.e. solving) an equation of motion modelling Coulomb and viscous frictional forces using a backward Euler approximation, the equation of motion having inputs of the calculated resultant force which acts on the first part of the surgical robot arm; and the current velocity at the current time of the first part of the surgical robot arm; and drive the surgical robot arm in accordance with the calculated desired velocity.

According to a second embodiment, there is provided a surgical system comprising a surgical robot arm and a controller according to the first embodiment.

Evaluating the equation of motion may comprise evaluating a deadband function of a value based on: the current velocity at the current time of the first part of the surgical robot arm, and the calculated resultant force which acts on the first part of the surgical robot arm.

The deadband function may have a deadband region and the gradient of the deadband function outside the deadband region may be selected so as to model desired viscous frictional forces acting on the surgical robot arm.

The limits of the deadband region of the deadband function may be selected so as to model desired Coulomb frictional forces acting on the surgical robot arm.

The equation of motion may be in accordance with:

$$M {\ddot{x}}_{k + 1} + {H}_{{F}_{C}}^{- 1} ( D {\dot{x}}_{k + 1} ) = f$$

where f represents the resultant force acting on the first part of the surgical robot arm;

$${H}_{{F}_{C}}^{- 1}$$

represents the inverse of the deadband function; \dot{x}ₖ₊₁ represents the desired velocity of the first part of the surgical robot arm at the time subsequent to the current time; \dot{x}ₖ₊₁ represents the acceleration of the first part of the surgical robot arm at the time subsequent to the current time; and M and D are predetermined constants.

D may be selected so as to model desired viscous frictional forces acting on the surgical robot arm.

Determining a torque at each of the plurality of joints may comprise: receiving a measurement of the torque at each of the plurality of joints from the surgical robot arm; and processing the torque measurements to account for torques which do not result from the external force imparted on the second part of the surgical robot arm.

The controller may be configured to receive a measurement of the position of the first part of the surgical robot arm from the surgical robot arm; and calculate from the measurement of the position, the current velocity at the current time of the first part of the surgical robot arm.

The backward Euler approximation may approximate the acceleration of the first part of the surgical robot arm at the time subsequent to the current time as the difference between the current velocity and the desired velocity of the first part of the surgical robot arm divided by the difference between the current time and the time subsequent to the current time.

The backward Euler approximation may be in accordance with:

$${\ddot{x}}_{k + 1} = \frac{{\dot{x}}_{k + 1} - {\dot{x}}_{k}}{{t}_{k + 1} - {t}_{k}}$$

where \ddot{x}ₖ₊₁ is the acceleration of the first part of the surgical robot arm at the time subsequent to the current time; \dot{x}ₖ₊₁ is the desired velocity; \dot{x}ₖ is the current velocity; tₖ is the current time; and tₖ₊₁ is the time subsequent to the current time.

The equation of motion may be in accordance with:

$${\dot{x}}_{k + 1} = ( \frac{{t}_{k + 1} - {t}_{k}}{D ( {t}_{k + 1} - {t}_{k} ) + M} ) {H}_{{F}_{c}} ( ( \frac{M}{{t}_{k + 1} - {t}_{k}} ) {\dot{x}}_{k} + f )$$

where tₖ represents the current time; tₖ₊₁ represents the time subsequent to the current time; \dot{x}ₖ₊₁ represents the desired velocity of the first part of the surgical robot arm at time tₖ₊₁; \dot{x}ₖ represents the current velocity of the first part of the surgical robot arm at the current time tₖ; H_{F}_{_{c}} represents the deadband function; f represents the resultant force acting on the first part of the surgical robot arm; and M and D are predetermined constants.

Driving the surgical robot arm in accordance with the calculated desired velocity may comprise: calculating from the desired velocity a desired position of the first part of the surgical robot arm; determining an angle of each of the plurality of joints which would allow the first part of the surgical robot arm to have the desired position; and sending a signal which causes the surgical robot arm to drive the plurality of joints to the determined angles.

The first part of the surgical robot arm may be an arm segment of the surgical robot arm.

The first part of the surgical robot arm may be the most distal arm segment of the surgical robot arm.

The first part of the surgical robot arm may be a joint of the plurality of driven joints of the surgical robot arm.

The first part of the surgical robot arm may be the elbow of the surgical robot arm.

The first part of the surgical robot arm may be the wrist of the surgical robot arm.

The second part of the surgical robot arm may be an arm segment of the surgical robot arm.

The second part of the surgical robot arm may be a joint of the plurality of driven joints of the surgical robot arm.

The first part and the second part may be the same part of the surgical robot arm.

The first part and the second part may be different parts of the surgical robot arm.

The external force imparted on the second part of the surgical robot arm may be a rotational force and the resultant force which acts on the first part of the surgical robot arm as a result of the external force may be a rotational force.

The surgical robot arm may comprise a torque sensor at each joint of the plurality of joints of the surgical robot arm and the surgical robot arm may be configured to measure the torque at each of the plurality of joints and send the measurements to the controller.

The surgical robot arm may comprise a position sensor at a plurality of points along the surgical robot arm and the surgical robot arm may be configured to measure the position of the first part of the surgical robot arm and send the measurement to the controller.

According to a third embodiment, there is provided a method for moving a first part of a surgical robot arm, the surgical robot arm comprising a plurality of arm segments separated by a plurality of joints, in response to an external force being imparted on a second part of the surgical robot arm, the method comprising: determining a torque at each of the plurality of joints as a result of the force imparted on the second part of the surgical robot arm; calculating from the determined torques a resultant force which acts on the first part of the surgical robot arm as a result of the external force being imparted on the second part of the surgical robot arm; calculating a desired velocity of the first part of the surgical robot arm for a time subsequent to a current time by evaluating (i.e. solving) an equation of motion modelling Coulomb and viscous frictional forces using a backward Euler approximation, the equation of motion having inputs of: the calculated resultant force which acts on the first part of the surgical robot arm; and the current velocity at the current time of the first part of the surgical robot arm; and driving the surgical robot arm in accordance with the calculated desired velocity.

According to a fourth embodiment, there is provided a non-transitory computer readable storage medium having stored thereon computer readable instructions that, when executed at a computer system, cause the computer system to perform a method according to the third embodiment.

### BRIEF DESCRIPTION OF THE FIGURES

FIG. 1 illustrates a surgical system.

FIG. 2 illustrates a function

$${H}_{y}^{- 1} ( D \dot{x} ) .$$

FIG. 3 illustrates a function

$${H}_{{F}_{C}}^{- 1} ( D \dot{x} )$$

corresponding to Coulomb and viscous friction.

FIG. 4 illustrates a function H_{Fc}(D\dot{x}).

FIG. 5 illustrates a method which the surgical system is configured to perform to implement the Coulomb and viscous friction model.

FIG. 6 illustrates a method which the surgical system is configured to perform to drive the surgical robot arm.

### DETAILED DESCRIPTION

It has previously been found to be effective to use a mass, spring, damper impedance model such that in response to an input external force, a robot arm behaves as if it is a very stiff spring in accordance with chosen mass, spring, damper coefficients. The mass, spring, damper coefficients are chosen such that the robot arm behaves as a very stiff spring at close to its maximum extension. According to the model, the setpoint of the spring is moved so as to maintain the spring extension at its maximum and the coefficients are chosen such that maximum extension of the spring is very small. The mass, spring damper model has been found to be very effective at allowing robot arms across a range of applications to exhibit desired behaviour. However, it has been found that for the application of robot arms for surgery, an alternative approach is advantageous.

Robot arms used in surgery require a far higher level of stability than robot arms used in other applications (e.g. manufacturing) due to the fact that the instrument attached to the arm is required to perform intricate and precise operations within a surgical site inside a human body. Since the instrument attached to the arm could be, for example a knife or cauteriser, it is critical that when the arm is instructed to keep the instrument stationary within the surgical site, its position does not change.

It has been found that the use of the mass, spring, damper model results in poor stability margins for amplitudes of motion below or near to the maximum spring extension, meaning that the position of the arm can oscillate within these bounds. The model can be tuned by changing the mass, spring, damper coefficients such that the oscillatory motion is very small and often imperceptible, however this can result in the modelled setpoint of the spring drifting over long periods of time. The result of this is that the position of parts of the arm (e.g. the terminal arm segment attached to the instrument) change when it is desirable that they remain stationary.

The present application concerns an alternative model to be used in controlling motion of the surgical robot arm, referred to herein in as a Coulomb and viscous friction model. The behaviour exhibited by the surgical robot arm as a result of implementing this model is analogous to that of an object sliding over another object in a stick-slip condition. The use of this model thus allows the arm to move in response to an external force being imparted on the arm as if it has a surface in close contact with and moving relative to another surface.

The technique used to implement a Coulomb and viscous friction model as described below may be used to dictate the movement of any part of the surgical robot arm. Based on the external force exerted on the surgical robot arm, the part of the robot arm which the force was intended to move (referred to herein as the first part of the surgical robot arm or first arm part) is deduced and the model is used to calculate the desired behaviour of the said first part. The term part is used herein to refer to any portion of the surgical robot arm. The first part of the surgical robot arm may be a part or all of an arm segment 105, a joint 104 or a combination of one or more arm segments 105 and/or joints 104.

The technique used to implement a Coulomb and viscous friction model described may also be used when an external force is applied to any part of the surgical robot arm 102. The part of the robot arm to which the force is applied may be, for example, an arm segment 105 or a joint 104. For example, the external force may be applied to the elbow joint 104e, the wrist joint 104w or to the terminal arm segment 105t to which the instrument is attached. The part of the robot arm to which the force is applied may equally be, for example, a portion of an arm segment 105, several arm segments 105, several joints 105 or a combination of one or more arm segments 105 and/or joints 104. The part of the robot arm to which the external force is imparted is referred to herein as the second part of the surgical robot arm or second arm part.

According to a first example (Example 1), a member of the bedside team may impart a force on a part of the arm with the intention of moving the same part of the arm. For example, the member of the bedside team may push on the elbow hoping to move the elbow to a different location. In such scenarios, the first part of the surgical robot arm is the same as the second part of the surgical robot arm.

However, according to a second example (Example 2) in which the instrument is positioned within a surgical site and the member of the bedside team is wanting to the change the instrument attached to the distal arm segment 105t, the member of the bedside team may exert a force on another of the arm segments 102 with the intention of retracting the terminal arm segment 102t (and the instrument) from the surgical site. According to this example, the first part of the surgical robot arm is different from the second part of the surgical robot arm.

As will be explained in more detail below, implementing the Coulomb and viscous friction model involves determining a resultant force which acts on the first part of the robot arm as a result of the external force being imparted on the second part of the robot arm. Using the second example above, the method would involve determining how much of the force imparted on another of the arm segments 102 contributes to a force acting on the terminal arm segment 102t.

As will be explained in more detail below, implementing the Coulomb and viscous friction model involves defining an equation of motion according to the model and solving it to obtain a desired velocity at which the first arm part should move at a future time so as to exhibit the desired behaviour. The current time is referred to herein as tₖ while the future time at which the first arm part will move at the desired velocity is referred to as tₖ₊₁. Implementing the Coulomb and viscous friction model therefore involves solving the equation of motion to obtain a value for the desired velocity \dot{x}ₖ₊₁.

The model dictates that a frictional force acts on the surface of the surgical robot arm in the opposite direction to that of the external force imparted on the arm. The frictional force is modelled using the Coulomb and viscous friction model as having two components: a Coulomb friction (F_{c}) component and a viscous friction (Fᵥ) component.

The viscous friction component (Fᵥ) is modelled as being proportional to velocity. In other words, the viscous friction component has a magnitude that is proportional to speed and acts in a direction against the direction of the velocity. The Coulomb friction component (F_{c}) is modelled as having a constant magnitude and acts in a direction against the direction of the velocity. The Coulomb friction component (F_{c}) is modelled as being equal to the static friction acting between the two surfaces. The Coulomb friction component (F_{c}) is non-zero.

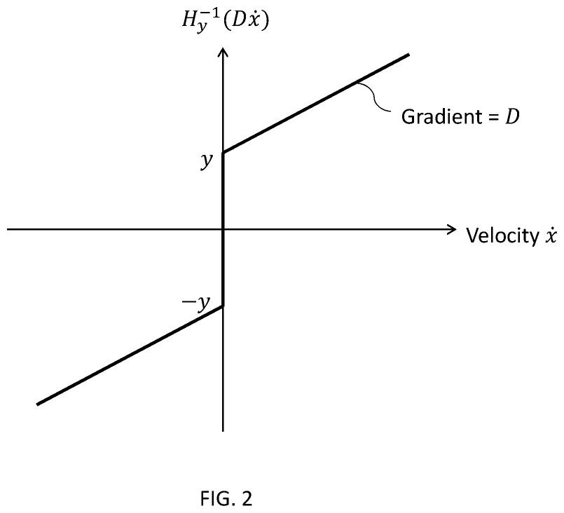

FIG. 2 illustrates a function

$${H}_{y}^{- 1} ( D \dot{x} )$$

which is the sum or a component that is proportional to velocity and a component with a constant magnitude of y in a direction against the direction of velocity. When the velocity is positive, the constant component has a value of y. When the velocity is negative, the constant component has a value of −y. FIG. 2 shows that the function

$${H}_{y}^{- 1} ( D \dot{x} )$$

takes the form of an inverse deadband function, which has an infinite gradient at \dot{x}=0 between the limits

$${H}_{y}^{- 1} ( D \dot{x} ) = y and {H}_{y}^{- 1} ( D \dot{x} ) = - y$$

and sloped portions on either side of \dot{x}=0 having a gradient equal to D. A deadband function is defined herein as encompassing any function which has a region having a gradient of 0. An inverse deadband function is defined herein as encompassing any function which has a region having an infinite gradient.

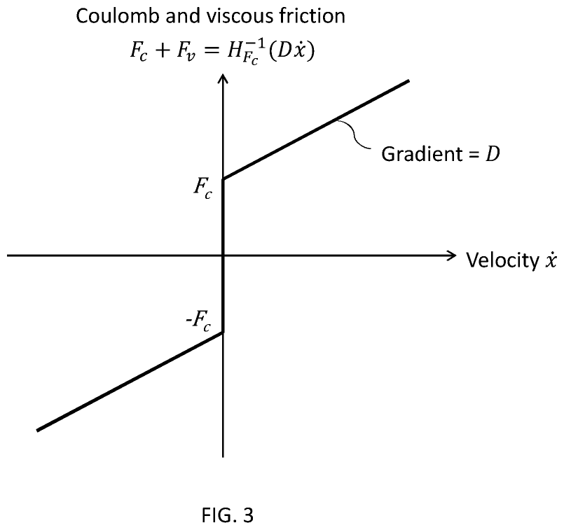

The sum of the Coulomb and viscous frictional forces can be represented using a function having the form of the function shown in FIG. 2. In particular, FIG. 3 illustrates the Coulomb and viscous frictional forces (F_{c}+Fᵥ) plotted against velocity \dot{x}.

The sum of the two components results in a function which looks the same as the inverse deadband function shown in FIG. 2, except that the value y has been replaced with the Coulomb frictional force F_{c}. The sum of the Coulomb and viscous frictional components can therefore be written as follows:

$${F}_{v} + {F}_{c} = {H}_{{F}_{c}}^{- 1} ( D \dot{x} ) \qquad ( equation 1 )$$

where D is a constant representing the gradient of the regions of the inverse deadband function away from \dot{x}=0.

The value of constant D may be chosen so as to model the desired viscous frictional force.

The value of F_{c} is selected so as to model desired Coulomb frictional forces acting on the surgical robot arm.

As previously explained, implementing the Coulomb and viscous friction model involves solving an equation of motion defined according to the model to determine a desired velocity at which the first arm part should move to achieve the desired behaviour.

At time tₖ₊₁, which is the point in time subsequent to the current time, at which the first arm part will move at the desired velocity \dot{x}ₖ₊₁, the equation of motion corresponding to the Coulomb and viscous friction model can be written as:

$$M {\ddot{x}}_{k + 1} + {H}_{{F}_{c}}^{- 1} ( D {\dot{x}}_{k + 1} ) = f \qquad ( equation 2 )$$

Where f is the resultant force acting on the first arm part, M is a constant representing the mass which the first arm part is modelled to have, \ddot{x}ₖ₊₁ is the acceleration of the first arm part at time

$$t = {t}_{k + 1} , {H}_{{F}_{c}}^{- 1} ( )$$

is the inverse deadband function, D is the constant representing the gradient of the regions of

$${H}_{{F}_{c}}^{- 1} ( )$$

away from \dot{x}=0 and \dot{x}ₖ₊₁ the desired velocity of the first arm part at time t=tₖ₊₁.

As previously explained, implementing the Coulomb and viscous friction model involves solving an equation of motion corresponding to the model (for example, equation 2) to obtain a desired velocity of the first arm part which will allow the arm to exhibit the desired behaviour in response to the external force exerted on the arm.

According to the present example, solving the equation of motion (equation 2) involves the use of a backward Euler approximation.

The backward Euler approximation approximates acceleration by starting from the subsequent time tₖ₊₁ and looking at the difference between the velocity at the subsequent time \dot{x}ₖ₊₁ and the previous velocity \dot{x}ₖ at the earlier (in this example, current) time tₖ.

The backward Euler approximation approximates the acceleration of the first arm part at the time subsequent to the current time tₖ₊₁ as the difference between the current velocity \dot{x}ₖ and the desired velocity \dot{x}ₖ₊₁ of the first arm part divided by the difference between the current time tₖ and the time subsequent to the current time tₖ₊₁.

The backward Euler approximation can be written as:

$${\ddot{x}}_{k + 1} = \frac{{\dot{x}}_{k + 1} - {\dot{x}}_{k}}{{t}_{k + 1} - {t}_{k}} \qquad ( equation 3 )$$

Inserting the backward Euler approximation into the equation of motion corresponding to the Coulomb and viscous friction model (equation 2) results in:

$$M ( \frac{{\dot{x}}_{k + 1} - {\dot{x}}_{k}}{{t}_{k + 1} - {t}_{k}} ) + {H}_{{F}_{c}}^{- 1} ( D {\dot{x}}_{k + 1} ) = f \qquad ( equation 4 )$$

which can be rearranged as:

$$( \frac{M}{{t}_{k + 1} - {t}_{k}} ) {\dot{x}}_{k + 1} + {H}_{{F}_{c}}^{- 1} ( D {\dot{x}}_{k + 1} ) = ( \frac{M}{{t}_{k + 1} - {t}_{k}} ) {\dot{x}}_{k} + f \qquad ( equation 5 )$$

It can be reasoned from the graphs shown in FIGS. 2 and 3, or algebraically, that:

$${H}_{{F}_{c}}^{- 1} ( ( D + \frac{M}{{t}_{k + 1} - {t}_{k}} ) {\dot{x}}_{k + 1} ) = ( \frac{M}{{t}_{k + 1} - {t}_{k}} ) {\dot{x}}_{k} + f \qquad ( equation 6 )$$

which can be rearranged as:

$${\dot{x}}_{k + 1} = ( \frac{{t}_{k + 1} - {t}_{k}}{D ( {t}_{k + 1} - {t}_{k} ) + M} ) {H}_{{F}_{c}} ( ( \frac{M}{{t}_{k + 1} - {t}_{k}} ) {\dot{x}}_{k} + f ) \qquad ( equation 7 )$$

Equation 7 can be solved to obtain a value for the desired velocity \dot{x}ₖ₊₁.

As will be seen from equations 4 to 7, solving the equation of motion to obtain the desired velocity \dot{x}ₖ₊₁ involves inverting the inverse deadband function

$${H}_{{F}_{c}}^{- 1}$$

to obtain the function H_{F}_{_{c}}, which is a deadband function, and evaluating the function H_{F}_{_{c}}.

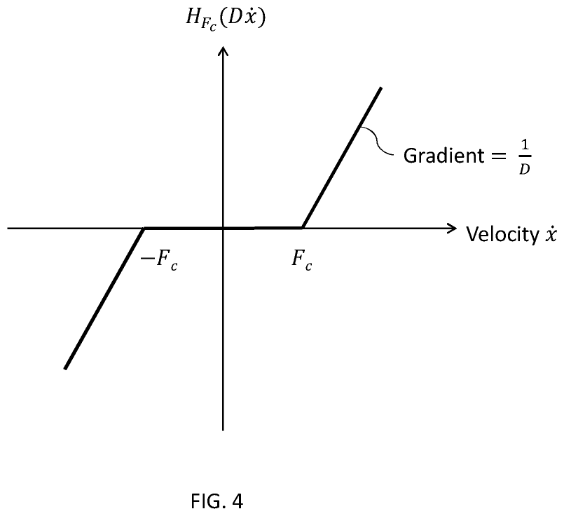

FIG. 4 illustrates the function H_{F}_{_{c}}(D\dot{x}). FIG. 4 shows that the function H_{F}_{_{c}}(D\dot{x}) comprises a portion (referred to herein as a deadband region) between the limits \dot{x}=−F_{c} and \dot{x}=F_{c} which has a value of zero (and a gradient of zero) and sloped portions on either side of the deadband region having a gradient equal to 1/D.

As will be seen from the graph shown in FIG. 4, evaluating the function H_{F}_{_{c}}(D\dot{x}) is straightforward due to the fact that the H_{F}_{_{c}}(D\dot{x}) is a continuous function.

Other approaches to solving the equation of motion (equation 2), such as the use of the forward Euler approximation, require evaluating the inverse deadband function

$${H}_{{F}_{C}}^{- 1} ,$$

which as seen in FIG. 3 is a discontinuous function. The output of the function

$${H}_{{F}_{C}}^{- 1} ( D \dot{x} )$$

at \dot{x}=0 is not well defined and can be any value within the range

$${H}_{{F}_{C}}^{- 1} ( D \dot{x} ) = {F}_{C} and {H}_{{F}_{C}}^{- 1} ( D \dot{x} ) = - {F}_{C} .$$

The result of evaluating the discontinuous function

$${H}_{{F}_{C}}^{- 1}$$

is therefore that the velocity \dot{x} will not rest at zero, but instead will be caused to oscillate around zero.

Solving the equation of motion (equation 2) using the backward Euler approximation as described herein is therefore advantageous as the problem of small oscillations which result from both the mass, spring, damper model and the forward Euler approximation are eliminated meaning that the stability of the surgical robot arm is improved.

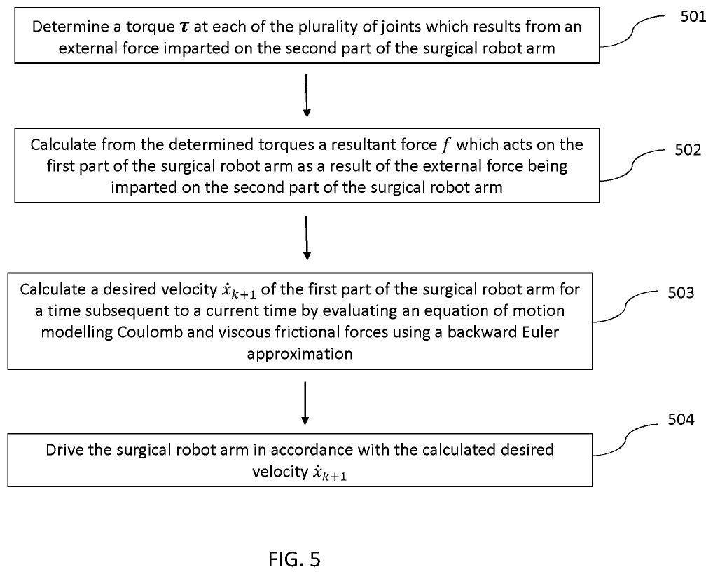

FIG. 5 illustrates the method which the controller 202 is configured to perform to implement the Coulomb and viscous friction model.

Step 501 shown in FIG. 5 states that the controller determines a torque at each of the plurality of joints which results from an external force imparted on the second part of the surgical robot arm.

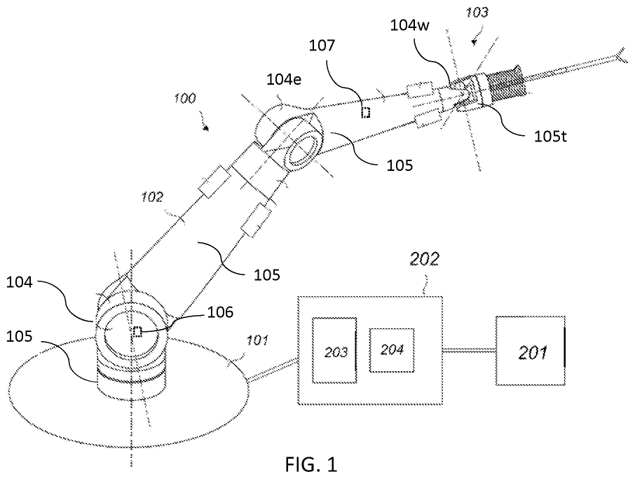

The surgical robot arm 102 illustrated in FIG. 1 comprises a torque sensor 106 located at each joint (not all shown). Each torque sensor is configured to measure the torque exerted on the joint at which it is located. The surgical robot arm 102 is configured to send the measurements of the torques exerted at the joints to the controller 202.

Step 501 of determining a torque at each of the plurality of joints which results from a force imparted on the second part of the surgical robot arm may comprise receiving a measurement of the torque at each of the plurality of joints (as measured by the torque sensors 106) from the surgical robot arm.

Step 501 may also comprise processing the received torque measurements and processing the received measurements so as to account for torques which do not result from the external force being imparted on the second arm part. For example, the received torque measurements may be adjusted so as to remove torques resulting from gravity due to the mass of the robot arm, torques resulting from internal gear train stretching and/or reaction torques resulting from the instrument interacting with the port and surgical site. The result of said processing is only the torque which results from the external force imparted on the second arm part.

Once the controller has determined a torque τ at each of the plurality of joints which results from an external force imparted on the second part of the surgical robot arm, the controller moves onto Step 502.

Step 502 shown in FIG. 5 states that the controller calculates from the torques determined in Step 501 a resultant force f which acts on the first part of the surgical robot arm as a result of the external force being imparted on the second part of the surgical robot arm.

Calculating the resultant force f may comprise calculating a force vector f corresponding to the resultant force acting at a series of points along the robot arm using the torques τ determined in Step 501. The force vector f may be calculated by applying the principle of virtual work to the joint torques τ. The principle of virtual work states that moving a point P on the arm a distance ∂p\hat{x} (Where \hat{x} is a unit vector) requires a joint J to move by an angle ∂θ_{J}, then if a force is applied at point P in the direction of \hat{x}, the torque τ_{J} seen by the joint J is proportional to the displacement over θ_{J}. This can be written in a Jacobian matrix Jₚ.

$$τ = ( \begin{matrix}{τ}_{1} \\ ⋮ \\ {τ}_{n}\end{matrix} ) = ( \begin{matrix}\frac{\partial {p}_{x}}{\partial {θ}_{1}} & … & \frac{\partial {p}_{z}}{\partial {θ}_{1}} \\ ⋮ & ⋱ & ⋮ \\ \frac{\partial {p}_{x}}{\partial {θ}_{n}} & … & \frac{\partial {p}_{z}}{\partial {θ}_{n}}\end{matrix} ) f = {J}_{p} f \qquad ( equation 8 )$$

where τ is a vector of the torques acting at each of the n joints of the surgical robot arm, τ is the torque acting at each joint, ∂pₓ is the distance moved by a series of points along the robot arm in the direction x, ∂θₙ is the angle moved by the joint n and f is a force vector corresponding to the resultant force acting at the series of points along the robot arm. The Jacobian matrix Jₚ is a function of the geometry and pose of the arm.

The result of resolving in directions x, y and z is:

$$τ = ( \begin{matrix}{τ}_{1} \\ ⋮ \\ {τ}_{n}\end{matrix} ) = ( \begin{matrix}\frac{\partial {p}_{x}}{\partial {θ}_{1}} & … & \frac{\partial {p}_{z}}{\partial {θ}_{1}} \\ ⋮ & ⋱ & ⋮ \\ \frac{\partial {p}_{x}}{\partial {θ}_{n}} & … & \frac{\partial {p}_{z}}{\partial {θ}_{n}}\end{matrix} ) ( \begin{matrix}{\hat{d}}_{x} \\ {\hat{d}}_{y} \\ {\hat{d}}_{z}\end{matrix} ) f = {J}_{p} \hat{d} f = ( \begin{matrix}\frac{\partial {p}_{\hat{d}}}{\partial {θ}_{1}} \\ ⋮ \\ \frac{\partial {p}_{\hat{d}}}{\partial {θ}_{n}}\end{matrix} ) f = {J}_{p , \hat{d}} f \qquad ( equation 9 )$$

which can be rearranged to form:

$$f = K τ \qquad ( equation 10 )$$

where K is the inverse of J_{p,\hat{d}}.

Step 502 may therefore comprise calculating the Jacobian matrix of displacement and joint angular displacement J_{p,\hat{d}}, finding the inverse of the Jacobian K and multiplying it by the torques acting at each of the n joints of the surgical robot arm to calculate a vector f representing the resultant forces which act at a series of points along the robot.

The surgical robot arm 102 illustrated in FIG. 1 comprises a position sensor 107 located at a plurality of points along the surgical robot arm (not all shown). Each position sensor is configured to measure the position of the point on the surgical robot arm at which it is located. The surgical robot arm 102 is configured to send the measurements of position at the points along the arm to the controller 202. The controller may be configured to infer angles of each of the joints n from the measurements of position by the position sensors and knowledge of the geometry of the surgical robot arm. The Jacobian J_{P} may therefore be generated by the controller according to measurements received from the surgical robot arm. Step 502 may therefore comprise receiving a measurement of position from the surgical robot arm.

As explained above, the result of multiplying the vector of the torques acting at each of the n joints of the surgical robot arm τ by the inverse Jacobian K is a vector f corresponding to the resultant force acting at the series of points along the robot arm.

As previously described, the technique used to implement a Coulomb and viscous friction model may be used to dictate the movement of any part of the surgical robot arm. The controller deduces the part of the arm which the force exerted on the second arm part was intended to move (referred to herein as the first arm part). The information regarding the force which acts on the first arm part as a result of the external force being imparted on the second arm part is contained within the vector f calculated using equations 8 to 10. This information is extracted from the vector f (disregarding the forces acting on other parts of the arm) to determine the resultant force which acts on the first part of the surgical robot arm.

It is noted that steps 501 and 502 for calculating a resultant force f are known in the art, and a skilled person would know how to implement these steps for controlling a surgical robot arm. Once the controller has calculated a resultant force f which acts on the first part of the surgical robot arm as a result of the external force being imparted on the second part of the surgical robot arm, the controller moves onto Step 503.

Step 503 of FIG. 5 states that the controller is configured to calculate a desired velocity \dot{x}ₖ₊₁ of the first part of the surgical robot arm for a time subsequent to a current time, tₖ₊₁ by evaluating an equation of motion modelling Coulomb and viscous frictional forces using a backward Euler approximation. The equation of motion has inputs of the calculated resultant force which acts on the first part of the surgical robot arm f, and the current velocity at the current time of the first part of the surgical robot arm \dot{x}ₖ. Step 503 may comprise evaluating an equation of motion of the form given in equation 2.

As previously explained, the surgical robot arm 102 illustrated in FIG. 1 comprises a position sensor 107 located at a plurality of points along the surgical robot arm, each position sensor being configured to measure the position of the point on the surgical robot arm at which is located and send the measurements of position at the points along the arm to the controller 202. The controller 202 may be configured to receive a measurement of the position of the first part of the surgical robot arm from the surgical robot arm and calculate from the measurement of the position, the current velocity \dot{x}ₖ at the current time tₖ of the first part of the surgical robot arm.

The controller is configured to evaluate the equation of motion using a backward Euler approximation. The backward Euler approximation may be of the form given in equation 3. Evaluation of the equation of motion given in equation 2 may be carried out by manipulating the equation as shown by equations 4 to 7. The controller may be configured to calculate the desired velocity \dot{x}ₖ₊₁ of the first part of the surgical robot arm by solving an equation of the form given in equation 7.

Solving the equation of motion comprises evaluating

$${H}_{{F}_{c}} ( ( \frac{M}{{t}_{k + 1} - {t}_{k}} ) {\dot{x}}_{k} + f ) ,$$

i.e. evaluating the deadband function (H_{F}_{_{c}}) of a value based on the current velocity at the current time of the first part of the surgical robot arm and the calculated resultant force which acts on the first part of the surgical robot arm. The calculated resultant force f which acts on the first part of the surgical robot arm is taken from the result of the calculation performed at Step 502.

Once the controller has calculated a desired velocity \dot{x}ₖ₊₁ of the first part of the surgical robot arm for a time subsequent to a current time, the controller moves onto Step 504. Step 504 states that the controller is configured to drive the surgical robot arm 102 in accordance with the calculated desired velocity \dot{x}ₖ₊₁ calculated in Step 503. In other words, the controller controls the robot arm such that the first arm part achieves the desired velocity \dot{x}ₖ₊₁ at time t=tₖ₊₁. A skilled person would know how to implement Step 504.

Therefore, a skilled person would know how to implements steps 501, 502 and 504. It is Step 503 (the calculation of the desired velocity by evaluating an equation of motion modelling Coulomb and viscous frictional forces using a backward Euler approximation) that provides the primary improvement of the present disclosure.

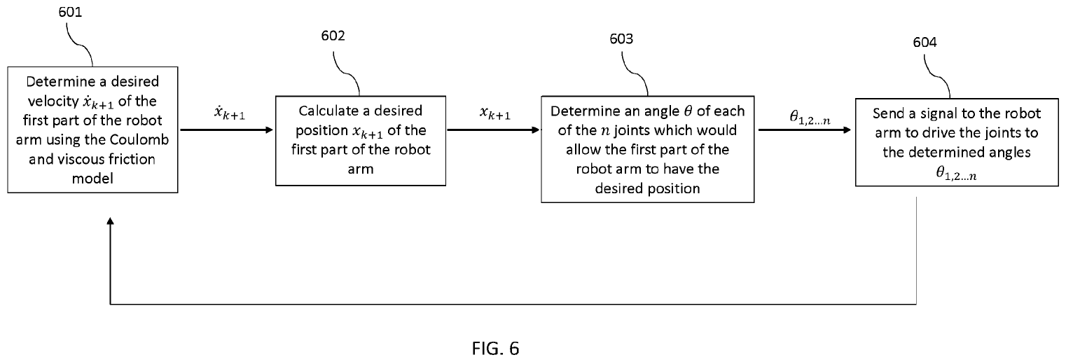

The controller may be configured to drive the surgical robot arm according to the method illustrated in FIG. 6. Step 601 of the method shown in FIG. 6 is to determine a desired velocity \dot{x}ₖ₊₁ of the first part of the robot arm using the Coulomb and viscous friction model. Step 601 may encompass all of Steps 501 to 503 seen in FIG. 6. Once a value for the desired velocity \dot{x}ₖ₊₁ has been determined, the controller moves onto Step 602. At Step 602, the controller calculates a desired position xₖ₊₁ of the first part of the arm. The desired position refers to the position the first arm part is desired to have at the time subsequent to the current time, tₖ₊₁. The desired position xₖ₊₁ may be calculated by integrating the desired velocity \dot{x}ₖ₊₁ with respect to time t and evaluating the position at t=tₖ₊₁. The controller may therefore be configured to calculate from the desired velocity a desired position of the first part of the surgical robot arm.

Once a value for desired position of the first arm part has been calculated, the controller moves onto Step 603. At Step 603, the controller determines an angle θ of each of the plurality of (n) joints of the robot arm which would define a pose allowing the first part of the arm to have the desired position xₖ₊₁. Determining the angles θ_{1,2 . . . n} may involve the use of inverse kinematics, as known in the art. Determining the angles θ_{1,2 . . . n} may require performing a calculation using a Jacobian matrix. Determining the angles θ_{1,2 . . . n} may require knowledge of the geometry of the arm, for example the length of each arm segment.

Once a value for the angle θ of each of the driven joints has been determined, the controller moves onto Step 604. At Step 604, the controller sends a signal to the robot arm which indicates that the joints should be driven to achieve the determined angles θ_{1,2 . . . n}. Sending a signal which causes the surgical robot arm to drive the plurality of joints to the determined angles may comprise sending a signal to actuators in the surgical robot arm such that the actuators rotate so as to drive the joints to the determined angles θ_{1,2 . . . n}.

The result of the controller implementing the method shown in FIG. 6 is that the first part of the robot arm is driven to the desired position such that the arm exhibits the desired behaviour as dictated by the Coulomb and viscous friction model.

As previously explained, this method can be applied to any part of the surgical robot arm so as to dictate the motion exhibited by said part of the arm. In some scenarios, such as the examples described below, implementing the Coulomb and viscous friction model may involve applying additional constraints so as to further control the behaviour exhibited by the robot arm.

According to Example 1 introduced above, a member of the bedside team may push on the elbow (thereby exerting an external force on a second arm part) hoping to move the elbow (a first arm part) to a different location. In such scenarios, the first part and the second part are the same part of the surgical robot arm. The resultant force f acting on the elbow according to this example may comprise only a linear component of force. If the force acts on the elbow while an instrument is attached to the terminal arm segment and is located within a surgical site, it is desirable that the position of the elbow can be moved as a result of an external force without affecting the position of the instrument within the surgical site. The Coulomb and viscous friction model in such a case may apply further constraints to the value of the desired velocity such that the desired velocity of the elbow may only be calculated to be a value which the elbow can achieve while maintaining the position of the instrument within the surgical site. This functionality can be facilitated due to the redundant nature of the surgical robot arm.

A related example defines a pivot point at the point on the instrument which is located at the entrance to the surgical site (i.e. the point on the instrument which is positioned just inside the port to the surgical site). In certain scenarios it may be desirable to change the position of the elbow in response to an external force without moving the pivot point. The Coulomb and viscous friction model in such a case may apply further constraints to the value of the desired velocity such that the desired velocity of the elbow may only be calculated to be a value which allows the instrument to pivot about the pivot point within the surgical site, but does not allow the pivot point on the instrument to move to another point on the instrument.

According to Example 2 introduced above, in which the instrument is positioned within a surgical site and the member of the bedside team is wanting to change the instrument attached to the distal arm segment 105t, the member of the bedside team may exert a force on an arm segment 102 adjacent to the terminal arm segment 105t with the intention of retracting the terminal arm segment 102t (and the instrument) from the surgical site. According to this example, the first part and the second part are different parts of the surgical robot arm. The resultant force f acting on the arm segment according to this example may comprise only a linear component of force. If the external force is imparted on the arm segment while the instrument attached to the terminal arm segment is located within a surgical site, it is desirable for the terminal arm segment to be extracted from the surgical site while following a path which is colinear with the instrument's longitudinal axis so as to minimise damage caused to the human body when the instrument is removed. The Coulomb and viscous friction model in such a case may apply further constraints to the value of the desired velocity of the terminal arm segment such that the desired velocity of the terminal arm segment may only be calculated as a velocity which acts in a direction along said longitudinal axis of the instrument.

According to other examples, the external force exerted on the surgical robot arm may comprise a linear component of force and a rotational component of force.

In a further example, the external force exerted on the arm may comprise only a rotational component of force. The external force imparted on the second part of the surgical robot arm may be solely a rotational force. The resultant force which acts on the first part of the surgical robot arm as a result of the external force may therefore also be solely a rotational force (i.e. a torque). This example may apply to scenarios in which a member of the bedside team grips the arm segment adjacent to the terminal arm segment 105t with the intention of rotating the instrument about its axis.

In such an example, Step 502 of calculating a resultant force may comprise calculating a resultant torque acting on the first arm part. Step 503 of calculating a desired velocity may comprise calculating a desired angular velocity \dot{θ}ₖ₊₁ of one or more joints by evaluating the equation of motion shown in equation 4 and replacing the calculated resultant force f with a calculated resultant torque t, the current velocity \dot{x}ₖ with a current angular velocity \dot{θ}ₖ and the “effective” mass M with an “effective” moment of inertia. Step 504 may therefore comprise driving the surgical robot arm in accordance with the calculated desired angular velocity \dot{θ}ₖ₊₁.

According to the same example, the method which is configured to be performed by the controller as set out in FIG. 6 to drive the arm may comprise Step 601 of determining a desired angular velocity \dot{θ}ₖ₊₁ of the first part of the robot arm using the Coulomb and viscous friction model. Step 602 may comprise calculating a desired angular position θₖ₊₁ of one or more joints and Step 603 may comprise determining an angle θ of each of the n joints other than the one or more joints so as to obtain an angle θ of each of the n joints of the robot arm (θ_{1,2 . . . n}) which would define a pose allowing the first part of the arm to have the desired angular position θₖ₊₁.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A controller configured to move a first part of a surgical robot arm, the surgical robot arm comprising a plurality of arm segments separated by a plurality of driven joints, in response to an external force being imparted on a second part of the surgical robot arm, the controller being configured to:
determine a torque at each of the plurality of joints which results from the force imparted on the second part of the surgical robot arm;
calculate from the determined torques a resultant force which acts on the first part of the surgical robot arm as a result of the external force being imparted on the second part of the surgical robot arm;
calculate a desired velocity of the first part of the surgical robot arm for a time subsequent to a current time by evaluating an equation of motion modelling Coulomb and viscous frictional forces using a backward Euler approximation, the equation of motion having inputs of:
the calculated resultant force which acts on the first part of the surgical robot arm; and
the current velocity at the current time of the first part of the surgical robot arm; and
drive the surgical robot arm in accordance with the calculated desired velocity.

2. The controller of claim 1, wherein evaluating the equation of motion comprises evaluating a deadband function of a value based on: the current velocity at the current time of the first part of the surgical robot arm, and the calculated resultant force which acts on the first part of the surgical robot arm.

3. The controller of claim 2, wherein the deadband function has a deadband region and the gradient of the deadband function outside the deadband region is selected so as to model desired viscous frictional forces acting on the surgical robot arm.

4. The controller of claim 3, wherein the limits of the deadband region of the deadband function are selected so as to model desired Coulomb frictional forces acting on the surgical robot arm.

5. The controller of claim 2, wherein the equation of motion is in accordance with:
$$M {\ddot{x}}_{k + 1} + {H}_{{F}_{C}}^{- 1} ( D {\dot{x}}_{k + 1} ) = f$$
where f represents the resultant force acting on the first part of the surgical robot arm;
$${H}_{{F}_{C}}^{- 1}$$
represents the inverse of the deadband function; \dot{x}ₖ₊₁ represents the desired velocity of the first part of the surgical robot arm at the time subsequent to the current time; \ddot{x}ₖ₊₁ represents the acceleration of the first part of the surgical robot arm at the time subsequent to the current time; and M and D are predetermined constants, wherein D is selected so as to model desired viscous frictional forces acting on the surgical robot arm.

6. (canceled)

7. The controller of claim 1, wherein determining a torque at each of the plurality of joints comprises:
receiving a measurement of the torque at each of the plurality of joints from the surgical robot arm; and
processing the torque measurements to account for torques which do not result from the external force imparted on the second part of the surgical robot arm.

8. The controller of claim 1, wherein the controller is configured to:
receive a measurement of the position of the first part of the surgical robot arm from the surgical robot arm; and
calculate from the measurement of the position, the current velocity at the current time of the first part of the surgical robot arm.

9. The controller of claim 1, wherein the backward Euler approximation approximates the acceleration of the first part of the surgical robot arm at the time subsequent to the current time as the difference between the current velocity and the desired velocity of the first part of the surgical robot arm divided by the difference between the current time and the time subsequent to the current time.

10. The controller of claim 1, wherein the backward Euler approximation is in accordance with:
$${\ddot{x}}_{k + 1} = \frac{{\dot{x}}_{k + 1} - {\dot{x}}_{k}}{{t}_{k + 1} - {t}_{k}}$$
where \ddot{x}ₖ₊₁ is the acceleration of the first part of the surgical robot arm at the time subsequent to the current time; \dot{x}ₖ₊₁ is the desired velocity; \dot{x}ₖ is the current velocity; tₖ is the current time; and tₖ₊₁ is the time subsequent to the current time.

11. The controller of claim 2, wherein the equation of motion is in accordance with:
$${\dot{x}}_{k + 1} = ( \frac{{t}_{k + 1} - {t}_{k}}{D ( {t}_{k + 1} - {t}_{k} ) + M} ) {H}_{{F}_{c}} ( ( \frac{M}{{t}_{k + 1} - {t}_{k}} ) {\dot{x}}_{k} + f )$$
where tₖ represents the current time; tₖ₊₁ represents the time subsequent to the current time; \dot{x}ₖ₊₁ represents the desired velocity of the first part of the surgical robot arm at time tₖ₊₁; \dot{x}ₖ represents the current velocity of the first part of the surgical robot arm at the current time tₖ; H_{F}_{_{c}} represents the deadband function; f represents the resultant force acting on the first part of the surgical robot arm; and M and D are predetermined constants.

12. The controller of claim 1, wherein driving the surgical robot arm in accordance with the calculated desired velocity comprises:
calculating from the desired velocity a desired position of the first part of the surgical robot arm;
determining an angle of each of the plurality of joints which would allow the first part of the surgical robot arm to have the desired position; and
sending a signal which causes the surgical robot arm to drive the plurality of joints to the determined angles.

13. The controller of claim 1, wherein the first part of the surgical robot arm is an arm segment of the surgical robot arm.

14. The controller of claim 13, wherein the first part of the surgical robot arm is the most distal arm segment of the surgical robot arm.

15. The controller of claim 1, wherein the first part of the surgical robot arm is a joint of the plurality of driven joints of the surgical robot arm.

16. (canceled)

17. (canceled)

18. The controller of claim 1, wherein the second part of the surgical robot arm is an arm segment of the surgical robot arm.

19. The controller of claim 1, wherein the second part of the surgical robot arm is a joint of the plurality of driven joints of the surgical robot arm.

20. The controller of claim 1, wherein the first part and the second part are the same part of the surgical robot arm or wherein the first part and the second part are different parts of the surgical robot arm.

21. (canceled)

22. The controller of claim 1, wherein the external force imparted on the second part of the surgical robot arm is a rotational force and the resultant force which acts on the first part of the surgical robot arm as a result of the external force is a rotational force.

23. (canceled)

24. (canceled)

25. (canceled)

26. A method for moving a first part of a surgical robot arm, the surgical robot arm comprising a plurality of arm segments separated by a plurality of joints, in response to an external force being imparted on a second part of the surgical robot arm, the method comprising:
determining a torque at each of the plurality of joints as a result of the force imparted on the second part of the surgical robot arm;
calculating from the determined torques a resultant force which acts on the first part of the surgical robot arm as a result of the external force being imparted on the second part of the surgical robot arm;
calculating a desired velocity of the first part of the surgical robot arm for a time subsequent to a current time by evaluating an equation of motion modelling Coulomb and viscous frictional forces using a backward Euler approximation, the equation of motion having inputs of:
the calculated resultant force which acts on the first part of the surgical robot arm; and
the current velocity at the current time of the first part of the surgical robot arm; and
driving the surgical robot arm in accordance with the calculated desired velocity.

27. A non-transitory computer readable storage medium having stored thereon computer readable instructions that, when executed at a computer system, cause the computer system to perform the method of claim 26.
